(19) United States
(12) Patent Application Publication
(10) Pub. No.: US 2007/0112210 A1
(43) Pub. Date: May 17, 2007

(54) Heterogeneous ruthenium catalyst, methods for hydrogenating a carbocyclic aromatic group, and nucleus-hydrogenated diglycidyl ether of bisphenols a and f

(75) Inventors: Jan-Dirk Arndt, Mannheim (DE); Frederik van Laar, Limburgerhof (DE); Michael Becker, Offenburg (DE)

Correspondence Address: CONNOLLY BOVE LODGE & HUTZ LLP, P.O. BOX 2207, WILMINGTON, DE, 19899-2207, US

(73) Assignee: BASF Aktiengesellschaft, Ludwigshafen (DE)

(21) Appl. No.: 10/583,543

(22) PCT Filed: Dec. 18, 2004
(86) PCT No.: PCT/EP04/14455

§ 371 Date: Jun. 19, 2006

(30) Foreign Application Priority Data
Dec. 22, 2003 (DE) 103 61 151.7
Nov. 18, 2004 (DE) 10 2004 055 805.1

(51) Int. Cl. B01J 23/40; C07D 405/02

(52) U.S. Cl. 549/555; 502/250

(57) ABSTRACT

Heterogeneous ruthenium catalyst comprising silicon dioxide as support material, wherein the catalyst surface comprises alkaline earth metal ions (M²⁺), process for hydrogenating a carbocyclic aromatic group to form the corresponding carbocyclic aliphatic group, in particular a process for preparing bisglycidyl ethers of the formula I 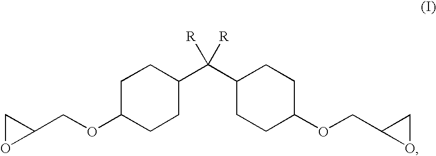
where R is CH₃ or H, by ring hydrogenation of the corresponding aromatic bisglycidyl ether of the formula II 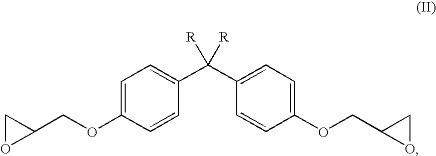 in which the abovementioned heterogeneous ruthenium catalyst is used, and bisglycidyl ethers of the formula I which can be prepared by the abovementioned process.

The present invention relates to a heterogeneous ruthenium catalyst comprising silicon dioxide as support material and a process for hydrogenating a carbocyclic aromatic group to form the corresponding carbocyclic aliphatic group, in particular a process for preparing a bisglycidyl ether of the formula I 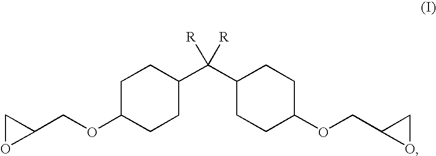
where R is CH₃ or H, by ring hydrogenation of the corresponding aromatic bisglycidyl ether of the formula II 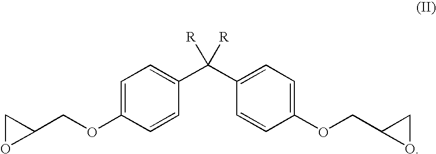
in the presence of a catalyst and bisglycidyl ethers of the formula I which can be prepared by this process.

The compound II in which R═H is also referred to as bis[glycidyloxyphenyl]methane (molecular weight: 312 g/mol).

The compound II in which R═CH₃ is also referred to as 2,2-bis[p-glycidyloxyphenyl]-propane (molecular weight: 340 g/mol).

The preparation of cycloaliphatic oxirane compounds I which contain no aromatic groups is of particular interest for the production of light- and weathering-resistant surface coating systems. Such compounds can in principle be prepared by hydrogenation of the corresponding aromatic compounds II. The compounds I are therefore also referred to as “ring-hydrogenated bisglycidyl ethers of bisphenols A and F”.

The compounds II have long been known as constituents of surface coating systems (cf. J. W. Muskopf et al. “Epoxy Resins” in Ullmann's Encyclopedia of Industrial Chemistry, 5th Edition on CD-ROM).

However, the high reactivity of the oxirane groups in the catalytic hydrogenation presents a problem. Under the reaction conditions usually required for the hydrogenation of the aromatic ring, these groups are frequently reduced to alcohols. For this reason, the hydrogenation of the compounds II has to be carried out under very mild conditions. However, this naturally results in a slowing of the desired aromatic hydrogenation.

U.S. Pat. No. 3,336,241 (Shell Oil Comp.) teaches the preparation of cycloaliphatic compounds containing epoxy groups by hydrogenation of corresponding aromatic epoxy compounds using rhodium and ruthenium catalysts. The activity of the catalysts decreases so much after one hydrogenation that the catalyst has to be changed after each hydrogenation in an industrial process. In addition, the selectivity of the catalysts described there leaves something to be desired.

DE-A-36 29 632 and DE-A-39 19 228 (both BASF AG) teach the selective hydrogenation of the aromatic parts of the molecule of bis[glycidyloxyphenyl]methane or of 2,2-bis[p-glycidyloxyphenyl]propane over ruthenium oxide hydrate. This improves the selectivity of the hydrogenation in respect of the aromatic groups to be hydrogenated. However, according to these teachings too, it is advisable to regenerate the catalyst after each hydrogenation, with the separation of the catalyst from the reaction mixture proving to present problems.

EP-A-678 512 (BASF AG) teaches the selective hydrogenation of the aromatic parts of the molecule of aromatic compounds containing oxirane groups over ruthenium catalysts, preferably ruthenium oxide hydrate, in the presence of from 0.2 to 10% by weight of water, based on the reaction mixture. Although the presence of water makes the separation of the catalyst from the reaction mixture easier, it does not alleviate the other disadvantages of these catalysts, e.g. an operating life which is in need of improvement.

EP-A-921 141 and EP-A1-1 270 633 (both Mitsubishi Chem. Corp.) concern the selective hydrogenation of double bonds in particular epoxy compounds in the presence of Rh and/or Ru catalysts having a particular surface area or in the presence of catalysts comprising metals of the platinum group.

JP-A-2002 226380 (Dainippon) discloses the ring hydrogenation of aromatic epoxy compounds in the presence of supported Ru catalysts and a carboxylic ester as solvent.

JP-A2-2001 261666 (Maruzen Petrochem.) relates to a process for the continuous ring hydrogenation of aromatic epoxide compounds in the presence of Ru catalysts which are preferably supported on activated carbon or aluminum oxide.

An article by Y. Hara et al. in Chem. Lett. 2002, pages 1116ff, relates to the “Selective Hydrogenation of Aromatic Compounds Containing Epoxy Group over Rh/Graphite”.

Tetrahedron Lett. 36, 6, pages 885-88, describes the stereoselective ring hydrogenation of substituted aromatics using colloidal Ru.

JP 10-204002 (Dainippon) relates to the use of specific Ru catalysts, in particular Ru catalysts doped with alkali metal, in ring hydrogenation processes.

JP-A-2002 249488 (Mitsubishi) teaches hydrogenation processes in which a supported noble metal catalyst having a chlorine content below 1500 ppm is used.

WO-A1-03/103 830 and WO-A1-04/009 526 (both Oxeno) relate to the hydrogenation of aromatic compounds, in particular the preparation of alicyclic polycarboxylic acids or esters thereof by ring hydrogenation of the corresponding aromatic polycarboxylic acids or esters thereof, and also to catalysts suitable for this purpose.

The processes of the prior art have the disadvantage that the catalysts used have only short operating lives and generally have to be regenerated in a costly fashion after each hydrogenation. The activity of the catalysts also leaves something to be desired, so that only low space-time yields, based on the catalyst used, are obtained under the reaction conditions required for a selective hydrogenation. However, this is not economically justifiable in view of the high cost of ruthenium and thus of the catalyst.

EP-A2-814 098 (BASF AG) relates to, inter alia, processes for the ring hydrogenation of organic compounds in the presence of specific supported Ru catalysts.

WO-A2-02/100 538 (BASF AG) describes a process for preparing particular cycloaliphatic compounds which have side chains containing epoxide groups by heterogeneously catalytic hydrogenation of a corresponding compound which contains at least one carbocyclic, aromatic group and at least one side chain containing at least one epoxide group over a ruthenium catalyst.

The ruthenium catalyst is obtainable by
i) treating a support material based on amorphous silicon dioxide one or more times with a halogen-free aqueous solution of a low molecular weight ruthenium compound and subsequently drying the treated support material at a temperature below 200° C.,
ii) reducing the solid obtained in i) by means of hydrogen at a temperature in the range from 100 bis 350° C.,
   with step ii) being carried out immediately after step i).

WO-A2-021100538 teaches that the compounds used can “be either monomeric compounds or oligomeric or polymeric compounds” (page 9 top).

WO-A2-02/100538 teaches nothing about the addition of alkaline earth metal ions.

It was an object of the present invention to provide an improved selective process for the hydrogenation of aromatic groups to the corresponding “ring-hydrogenated” groups, by means of which high yields and space-time yields [amount of product/(catalyst volume·time)] (kg/(l ·h)), [amount of product/(reactor volume·time)] (kg/(l_{reactor}·h)), based on the catalyst used, can be achieved and in which the catalysts used can be used for hydrogenations a number of times without work-up. In particular, catalyst operating lives which are higher than those in the process of WO-A2-02/100 538 should be achieved. Furthermore, bisglycidyl ethers of the formula I having improved properties, in particular in their typical applications, are to be found.

We have accordingly found a heterogeneous ruthenium catalyst comprising silicon dioxide as support material, wherein the catalyst surface comprises alkaline earth metal ions (M²⁺), and a process for hydrogenating a carbocyclic aromatic group to form the corresponding carbocyclic aliphatic group, in particular a process for preparing the bisglycidyl ethers of the formula I 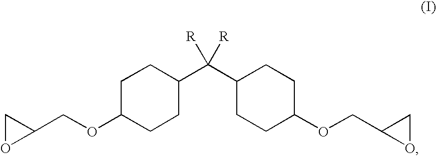
where R is CH₃ or H, by ring hydrogenation of the corresponding aromatic bisglycidyl ether of the formula II 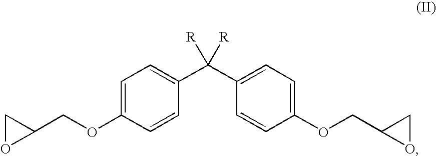
wherein the abovementioned heterogeneous ruthenium catalyst is used, and bisglycidyl ethers of the formula I which can be prepared by the abovementioned process.

An important constituent of the catalysts of the invention is the support material based on amorphous silicon dioxide. In this context, the term “amorphous” means that the proportion of crystalline silicon dioxide phases in the support material is less than 10% by weight. However, the support material used for producing the catalysts can display superstructures formed by a regular arrangement of pores in the support material.

The catalyst surface of the catalysts of the invention comprises alkaline earth metal ions (M²⁺), i.e. M=Be, Mg, Ca, Sr and/or Ba, in particular Mg and/or Ca, very particularly preferably Mg.

Possible support materials are basically amorphous silicon dioxides comprising at least 90% by weight of silicon dioxide, with the remaining 10% by weight, preferably not more than 5% by weight, of the support material also being able to be another oxidic material, e.g. MgO, CaO, TiO₂, ZrO₂, Fe₂O₃ and/or an alkali metal oxide.

In a preferred embodiment of the invention, the support material is halogen-free, in particular chlorine-free, i.e. the halogen content of the support material is less than 500 ppm by weight, for example in the range from 0 to 400 ppm by weight.

Preference is given to support materials which have a specific surface area in the range from 30 to 700 m²/g, preferably from 30 to 450 m²/g (BET surface area in accordance with DIN 66131).

Suitable amorphous support materials based on silicon dioxide are well known to those skilled in the art and are commercially available (cf., for example, O. W. Flörke, “Silica” in Ullmann's Encyclopedia of Industrial Chemistry 6th Edition on CD-ROM). They can either be of natural origin or have been produced synthetically. Examples of suitable amorphous support materials based on silicon dioxide are silica gels, kieselguhr, pyrogenic silicas and precipitated silicas. In a preferred embodiment of the invention, the catalysts have silica gels as support materials.

Depending on the way in which the invention is performed, the support material can have various forms. If the process is carried out as a suspension process, the support material will usually be used in the form of finely divided powder for producing the catalysts of the invention. The powder preferably has particle sizes in the range from 1 to 200 μm, in particular from 1 to 100 μm. When the catalyst is used in fixed beds, it is usual to employ shaped bodies made of the support material which are obtainable, for example, by extrusion, ram extrusion or tableting and can have, for example, the shape of spheres, pellets, cylinders, extrudates, rings or hollow cylinders, stars and the like. The dimensions of these shaped bodies are usually in the range from 1 mm to 25 mm. Catalyst extrudates having extrudate diameters of from 1.5 to 5 mm and extrudate lengths of from 2 to 25 mm are frequently used.

The ruthenium content in the catalysts can be varied over a wide range. It will preferably be at least 0.1% by weight, advantageously at least 0.2% by weight, and will frequently not exceed a value of 10% by weight, in each case based on the weight of the support material and calculated as elemental ruthenium. The ruthenium content is preferably in the range from 0.2 to 7% by weight, in particular in the range from 0.4 to 5% by weight, e.g. from 1.5 to 2% by weight.

The content of alkaline earth metal ion(s) (M²⁺) in the catalyst surface is preferably from 0.01 to 1% by weight, in particular from 0.05 to 0.5% by weight, very particularly preferably from 0.1 to 0.25% by weight, in each case based on the weight of the silicon dioxide support material.

The ruthenium catalysts of the invention are preferably produced by firstly treating the support material with a solution of a low molecular weight ruthenium compound, hereinafter referred to as (ruthenium) precursor, in such a way that the desired amount of ruthenium is taken up by the support material. Preferred solvents here are glacial acetic acid, water or mixtures thereof. This step will hereinafter also be referred to as impregnation. The support which has been treated in this way is subsequently dried, preferably with the upper limit to the temperature mentioned below being adhered to. If appropriate, the solid obtained in this way is then treated again with the aqueous solution of the ruthenium precursor and dried again. This procedure is repeated until the amount of ruthenium compound taken up by the support material corresponds to the desired ruthenium content of the catalyst.

The treatment or impregnation of the support material can be carried out in various ways and depends in a known manner on the shape of the support material. For example, the support material can be sprayed or flushed with the precursor solution or the support material can be suspended in the precursor solution. For example, the support material can be suspended in the aqueous solution of the ruthenium precursor and filtered off from the aqueous supernatant liquid after a particular time. The ruthenium content of the catalyst can then be controlled in a simple fashion via the amount of liquid taken up and the ruthenium concentration of the solution. The impregnation of the support material can, for example, also be carried out by treating the support with a defined amount of the solution of the ruthenium precursor corresponding to the maximum amount of liquid which can be taken up by the support material. For this purpose, the support material can, for example, be sprayed with the required amount of liquid. Suitable apparatuses for this purpose are the apparatuses customarily used for mixing liquids with solids (cf. Vauck/Müller, Grundoperationen chemischer Verfahrenstechnik, 10^{th} edition, Deutscher Verlag für Grundstoffindustrie, 1994, p. 405 ff.), for example tumble dryers, impregnation drums, drum mixers, blade mixers and the like. Monolithic supports are usually flushed with the aqueous solutions of the ruthenium precursor.

The solutions used for impregnation are preferably low in halogen, in particular low in chlorine, i.e. they comprise no halogen or less than 500 ppm by weight, in particular less than 100 ppm by weight, of halogen, e.g. from 0 to <80 ppm by weight of halogen, based on the total weight of the solution. Ruthenium precursors used are therefore RuCl₃ and preferably ruthenium compounds which comprise no chemically bound halogen and are sufficiently soluble in the solvent. These include, for example, ruthenium(III) nitrosyl nitrate (Ru(NO)(NO₃)₃), ruthenium(III) acetate and also alkali metal ruthenates(IV), e.g. sodium and potassium ruthenate(IV).

A very particularly preferred Ru precursor is Ru(III) acetate. This Ru compound is usually employed as a solution in acetic acid or glacial acetic acid, but it can also be used as a solid. The catalyst of the invention can be produced without using water.

Many ruthenium precursors are commercially available as solutions, but the corresponding solids can also be used. These precursors can be dissolved or diluted using the same component as the solvent supplied, e.g. nitric acid, acetic acid, hydrochloric acid, or preferably using water. Mixtures of water or solvent containing up to 50% by volume of one or more organic solvents which are miscible with water or solvents, e.g. mixtures with C₁-C₄-alkanols such as methanol, ethanol, n-propanol or isopropanol, can also be used. All mixtures should be chosen so that a single solution or phase is present. The concentration of the ruthenium precursor in the solutions naturally depends on the amount of ruthenium precursor to be applied and on the uptake capacity of the support material for the solution and is preferably in the range from 0.1 to 20% by weight.

Drying can be carried out by the customary methods of solids drying with the upper limits to the temperature mentioned below being adhered to. Adherence to the upper limit to the drying temperature is important for the quality, i.e. the activity, of the catalyst. Exceeding the drying temperatures mentioned below leads to a significant loss in activity. Calcination of the support at higher temperatures, e.g. above 300° C. or even 400° C., as is proposed in the prior art, is not only superfluous but also has an adverse effect on the activity of the catalyst. To achieve satisfactory drying rates, drying is preferably carried out at elevated temperature, preferably at ≦180° C., in particular at ≦160° C. and at least 40° C., in particular at least 70° C., especially at least 100° C., very particularly at least 140° C.

Drying of the solid impregnated with the ruthenium precursor is usually carried out under atmospheric pressure, although a reduced pressure can also be employed to promote drying. A gas stream, e.g. air or nitrogen, will frequently be passed over or through the material to be dried in order to promote drying.

The drying time naturally depends on the desired degree of drying and on the drying temperature and is preferably in the range from 1 hour to 30 hours, preferably in the range from 2 to 10 hours.

Drying of the treated support material is preferably carried out to the point where the content of water or of volatile solvent constituents prior to the subsequent reduction is less than 5% by weight, in particular not more than 2% by weight, based on the total weight of the solid. The proportions by weight indicated correspond to the weight loss of the solid determined at a temperature of 160° C., a pressure of 1 bar and a time of 10 minutes. In this way, the activity of the catalysts used according to the invention can be increased further.

Drying is preferably carried out with the solid which has been treated with the precursor solution being kept in motion, for example by drying the solid in a rotary tube oven or a rotary sphere oven. In this way, the activity of the catalysts of the invention can be increased further.

The conversion of the solid obtained after drying into its catalytically active form is achieved by reducing the solid in a manner known per se at the temperatures indicated above.

For this purpose, the support material is brought into contact with hydrogen or a mixture of hydrogen and an inert gas at the temperatures indicated above. The absolute hydrogen pressure is of minor importance for the result of the reduction and will be for example in the range from 0.2 bar to 1.5 bar. The hydrogenation of the catalyst material is frequently carried out at a hydrogen pressure of one atmosphere in a stream of hydrogen. The reduction is preferably carried out with the solid being kept in motion, for example by reducing the solid in a rotary tube oven or a rotary sphere oven. In this way, the activity of the catalysts of the invention can be increased further.

The reduction can also be carried out by means of organic reducing agents such as hydrazine, formaldehyde, formates or acetates.

After the reduction, the catalyst can be passivated in a known manner, e.g. by briefly treating the catalyst with an oxygen-containing gas, e.g. air, but preferably with an inert gas mixture comprising from 1 to 10% by volume of oxygen, to improve the handleability. CO₂ or CO₂/O₂ mixtures can also be employed here.

The active catalyst can also be stored under an inert organic solvent, e.g. ethylene glycol.

A preferred way of producing the catalyst of the invention comprises impregnating the ruthenium catalyst precursor, e.g. produced as above or as described in WO-A2-02/100538 (BASF AG), with a solution of one or more alkaline earth metal(II) salts.

Preferred alkaline earth metal(II) salts are corresponding nitrates, in particular magnesium nitrate and calcium nitrate.

A preferred solvent for the alkaline earth metal(II) salts in this impregnation step is water. The concentration of the alkaline earth metal(II) salt in the solvent is, for example, from 0.01 to 1 mol/liter.

For example, the Ru/SiO₂ catalyst installed in a tube is brought into contact with a stream of an aqueous solution of the alkaline earth metal salt. The catalyst to be impregnated can also be treated with a supernatant solution of the alkaline earth metal salt.

Preferably, the Ru/SiO₂ catalyst is in this way saturated, in particular at its surface, with the alkaline earth metal ion(s).

Excess alkaline earth metal salt and alkaline earth metal ions which have not been immobilized is/are rinsed from the catalyst (H₂O rinse, catalyst washing).

To simplify handling, e.g. installation in a reactor tube, the catalyst of the invention can be dried after impregnation. For this purpose, drying can be carried out in an oven at <200° C., e.g. from 50 to 190° C., particularly preferably at <140° C., e.g. at from 60 to 130° C.

This impregnation process can be carried out ex situ or in situ: ex situ means before installation of the catalyst in the reactor, in situ means in the reactor (after installation of the catalyst).

In one process variant, the impregnation of the catalyst surface with alkaline earth metal ions can also be carried out in situ by alkaline earth metal ions, e.g. in the form of dissolved alkaline earth metal salts, being added to the solution of the aromatic substrate to be hydrogenated (starting material). For this purpose, the appropriate amount of salt is, for example, firstly dissolved in water and then added to the substrate dissolved in an organic solvent.

The content of alkaline earth metal ions in the solution of the aromatic substrate to be hydrogenated is generally from 1 to 100 ppm by weight, in particular from 2 to 10 ppm by weight.

In one variant, it has been found to be particularly advantageous for the catalyst of the invention to be used in combination with a solution of the aromatic substrate to be hydrogenated which contains alkaline earth metal ions in the hydrogenation process of the invention.

As a result of the way in which the catalysts of the invention are produced, the ruthenium is present as metallic ruthenium in these catalysts. Furthermore, electron-microscopic studies (SEM or TEM) have shown that a surface-impregnated catalyst is obtained: the ruthenium concentration within a catalyst particle decreases from the outside toward the interior, with a ruthenium layer being present at the surface of the particle. In preferred cases, crystalline ruthenium can be detected in the outer layer by means of SAD (selected area diffraction) and XRD (X-ray diffraction).

In addition, as a result of the use of halogen-free, in particular chlorine-free, ruthenium precursors and solvents in the production of the catalysts of the invention, their halide content, in particular chloride content, is below 0.05% by weight (0 to <500 ppm by weight, for example in the range from 0-400 ppm by weight), based on the total weight of the catalyst.

The chloride content is determined by ion chromatography, for example by the method described below.

In this document, all ppm figures are by weight (ppm by weight) unless indicated otherwise.

In a selected variant, preference is given to the percentage ratio of the Q₂ and Q₃ structures Q₂/Q₃ determined by means of solid-state ²⁹Si-NMR being less than 25, preferably less than 20, particularly preferably less than 15, e.g. in the range from 0 to 14 or 0.1 to 13. This also means that the degree of condensation of the silica in the support used is particularly high.

The identification of the Qₙ structures (n=2, 3, 4) and the determination of the percentage ratio is carried out by means of solid-state ²⁹Si-NMR.
Qₙ=Si(OSi)ₙ(OH)₄₋ₙ where n=1, 2, 3 or 4.

Qₙ is found at −110.8 ppm when n=4, at −100.5 ppm when n=3 and at −90.7 ppm when n=2 (standard: tetramethylsilane) (Q₀ and Q₁ were not identified). The analysis is carried out under the conditions of “magic angle spinning” at room temperature (20° C.) (MAS 5500 Hz) with circular polarization (CP 5 ms) and using dipolar decoupling of ¹H. Owing to the partial superimposition of the signals, the intensities were evaluated via line shape analysis. The line shape analysis was carried out using a standard software package from Galactic Industries, with an iterative “least square fit” being calculated.

The support material preferably comprises no more than 1% by weight, in particular not more than 0.5% by weight and particularly preferably <500 ppm by weight, of aluminum oxide, calculated as Al₂O₃.

Since the condensation of the silica can also be influenced by aluminum and iron, the total concentration of Al(III) and Fe(II and/or III) is preferably less than 300 ppm, particularly preferably less than 200 ppm and is for example in the range from 0 to 180 ppm.

The alkali metal oxide content preferably results from the production of the support material and can be up to 2% by weight. It is frequently less than 1% by weight. Supports which are free of alkali metal oxide (0 to <0.1% by weight) are also suitable. The proportion of MgO, CaO, TiO₂ or ZrO₂ can amount to up to 10% by weight of the support material and is preferably not more than 5% by weight. However, support materials which contain no detectable amounts of these metal oxides (0 to <0.1% by weight) are also suitable.

Since Al(III) and Fe(II and/or III) incorporated in silica can produce acid centers, it is preferred that charge-compensating cations, preferably alkaline earth metal cations (M²⁺, M=Be, Mg, Ca, Sr, Ba), are present in the support. This means that the weight ratio of M(II) to (Al(III)+Fe(II and/or III)) is greater than 0.5, preferably >1, particularly preferably greater than 3.

The Roman numbers in brackets after the element symbol indicate the oxidation state of the element.

The carbocyclic aromatic group in the organic compound to be hydrogenated is in particular a benzene ring, which may bear substituents.

Examples of compounds containing a benzene ring which are able to be hydrogenated by the process of the invention to form the corresponding compound containing a saturated carbocyclic 6-membered ring are listed in the following table:

| Starting material | Product |
|---|---|
| Benzene | Cyclohexane |
| Toluene | Methylcyclohexane |
| Ethylbenzene | Ethylcyclohexane |
| Xylene (o-, m- or p-) or isomer mixture | Dimethylcyclohexane |
| Phenol | Cyclohexanol |
| Alkyl-substituted phenols, e.g. C₁₋₁₀- | Alkyl-substituted cyclohexanols, |
| alkylphenol, such as 4-tert-butylphenol, | e.g. C₁₋₁₀- |
| 4-nonylphenol | alkylcyclohexanol |
| Bis(p-hydroxyphenyl)methane | Bis(4- |
| | hydroxycyclohexyl)methane |
| Bis(p-hydroxyphenyl)dimethylmethane | Bis(4- |
| | hydroxycyclohexyl)dimethyl- |
| | methane |
| Aniline | Cyclohexylamine |
| C₁₋₁₀-Alkyl-substituted aniline | C₁₋₁₀-Alkyl- |
| | substituted cyclohexylamine |
| N,N-di-C₁₋₁₀-Alkylaniline | N,N-di-C₁₋₁₀- |
| | Alkylcyclohexylamine |
| Diaminobenzene | Diaminocyclohexane |
| Bis(p-aminophenyl)methane | Bis(4-aminocyclohexyl)methane |

As starting compounds for the hydrogenation process of the invention, mention may also be made by way of example of the following substance classes and materials:
reaction products of bisphenol A or bisphenol F or comparable alkylene- or cycloalkylene-bridged bisphenol compounds with epichlorohydrin.
Bisphenol A or bisphenol F or comparable compounds can be reacted with epichlorohydrin and bases in a known manner (cf. Ullmann's Encyclopedia of Industrial Chemistry, 5th Edition, VCH (1987), Vol. A9, p. 547) to give glycidyl ethers of the general formula IIa, 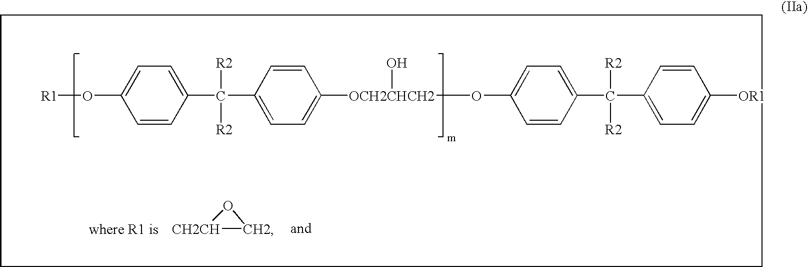
R² is hydrogen or a C₁-C₄-alkyl group, e.g. methyl, or two radicals R² bound to one carbon atom form a C₃-C₅-alkylene group, and m is from zero to 40.
Phenol and cresol epoxy novolaks IIb
Novolaks of the general formula IIb can be obtained by acid-catalyzed reaction of phenol and cresol and conversion of the reaction products into the corresponding glycidyl ethers (e.g. bis[4-(2,3-epoxypropoxy)phenyl]methane): 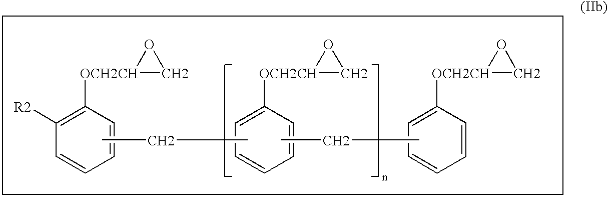
where R² is hydrogen or a methyl group and n is from 0 to 40 (cf. J. W. Muskopf et al. “Epoxy Resins 2.2.2” in Ullmann's Encyclopedia of Industrial Chemistry, 5th Edition on CD-ROM).
Glycidyl ethers of reaction products of phenol and an aldehyde:
Acid-catalyzed reaction of phenol and aldehydes and subsequent reaction with epichlorohydrin makes it possible to obtain glycidyl ethers, e.g. 1,1,2,2-tetrakis[4-(2,3-epoxypropoxy)phenyl]ethane can be obtained from phenol and glyoxal (cf. J. W. Muskopf et al. “Epoxy Resins 2.2.3” in Ullmann's Encyclopedia of Industrial Chemistry, 5th Edition on CD-ROM).
Glycidyl ethers of phenol-hydrocarbon novolaks, e.g. 2,5-bis[(glycidyloxy)phenyl]octahydro-4,7-methano-5H-indene and its oligomers.
Aromatic glycidyl amines:
Examples which may be mentioned are the triglycidyl compound of p-amino-phenol, 1-(glycidyloxy)-4-[N,N-bis(glycidyl)amino]benzene, and the tetraglycidyl compound of methylenediamine, bis{4-[N,N-bis(2,3-epoxypropyl)amino]-phenyl}methane.

Further specific examples are: tris[4-(glycidyloxy)phenyl]methane isomers and glycidyl esters of aromatic monocarboxylic, dicarboxylic and tricarboxylic acids, e.g. diglycidyl phthalates and isophthalates.

In a particular embodiment of the process of the invention, aromatic bisglycidyl ethers of the formula II 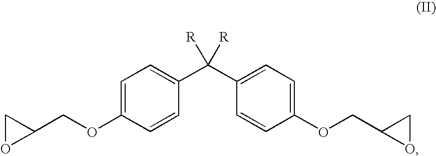
where R is CH₃ or H, are ring hydrogenated.

Preferred aromatic bisglycidyl ethers of the formula II have a content of chloride and/or organically bound chlorine of ≦1000 ppm by weight, particularly preferably <950 ppm by weight, in particular in the range from 0 to <800 ppm by weight, e.g. from 600 to 1000 ppm by weight.

The content of chloride and/or organically bound chlorine is, for example, determined ion-chromatographically or coulometrically using the methods described below.

According to a particular embodiment of this process variant according to the invention, it has been recognized that it is, surprisingly, also advantageous for the aromatic bisglycidyl ether of the formula II to have a content of corresponding oligomeric bisglycidyl ethers of less than 10% by weight, in particular less than 5% by weight, particularly preferably less than 1.5% by weight, very particularly preferably less than 0.5% by weight, e.g. in the range from 0 to <0.4% by weight.

According to this particular embodiment of this process variant according to the invention, it has been found that the oligomer content of the feed has a critical influence on the operating life of the catalyst, i.e. the conversion remains at a high level for longer. When a bisglycidyl ether II which has, for example, been distilled and is therefore low in oligomers is used, a slowed catalyst deactivation compared to a corresponding commercial standard product (e.g.: ARALDIT GY 240 BD from Vantico) is observed.

The oligomer content of the aromatic bisglycidyl ether of the formula II which is used is preferably determined by GPC measurement (gel permeation chromatography) or by determination of the evaporation residue.

The evaporation residue is determined by heating the aromatic bisglycidyl ether for 2 hours at 200° C. and for a further 2 hours at 300° C., in each case at 3 mbar.

For the further respective conditions for determining the oligomer content, see below.

The respective oligomeric bisglycidyl ethers generally have a molecular weight determined by GPC in the range from 380 to 1500 g/mol and possess, for example, the following structures (cf., for example, Journal of Chromatography 238 (1982), pages 385-398, page 387):

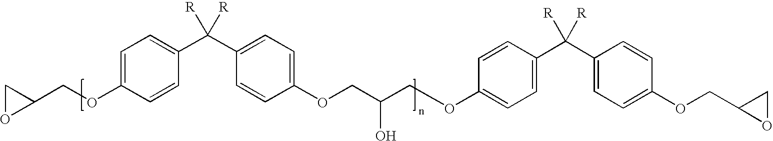

R═CH₃ or H. n=1,2, 3 or 4

The respective oligomeric bisglycidyl ethers have a molecular weight in the range from 568 to 1338 g/mol, in particular from 568 to 812 g/mol, when R═H, and have a molecular weight in the range from 624 to 1478 g/mol, in particular from 624 to 908 g/mol, when R═CH₃.

The removal of the oligomers is carried out, for example, by means of chromatography or, on a relatively large scale, preferably by distillation, e.g. in a batch distillation on the laboratory scale or in a thin film evaporator, preferably in a short path distillation, on an industrial scale, in each case under reduced pressure.

In a batch distillation for the removal of oligomers at, for example, a pressure of 2 mbar, the bath temperature is about 260° C. and the temperature at which the distillate goes over at the top is about 229° C.

Oligomer removal can likewise be carried out under milder conditions, for example under reduced pressures in the range from 1 to 10⁻³ mbar. At a working pressure 0.1 mbar, the boiling point of the oligomer-containing starting material is reduced by 20-30° C., depending on the starting material, and the thermal stress on the product is therefore also reduced. To minimize the thermal stress, the distillation is preferably carried out continuously in a thin film evaporation or particularly preferably in a short path evaporation.

In the process of the invention, the hydrogenation of the starting materials, e.g. the compounds II, preferably occurs in the liquid phase. The hydrogenation can be carried out in the absence of solvents or in an organic solvent. Owing to the sometimes high viscosity of the compounds II, they are preferably used as a solution or mixture in an organic solvent.

Possible organic solvents are basically those which are able to dissolve the starting material, e.g. the compound II, virtually completely or are completely miscible with this and are inert under the hydrogenation conditions, i.e. are not hydrogenated.

Examples of suitable solvents are cyclic and acyclic ethers, e.g. tetrahydrofuran, dioxane, methyl tert-butyl ether, dimethoxyethane, dimethoxypropane, dimethyl diethylene glycol, aliphatic alcohols such as methanol, ethanol, n-propanol or isopropanol, n-, 2-, iso- or tert-butanol, carboxylic esters such as methyl acetate, ethyl acetate, propyl acetate or butyl acetate, and also aliphatic ether alcohols such as methoxypropanol.

The concentration of starting material, e.g. of compound II, in the liquid phase to be hydrogenated can in principle be chosen freely and is frequently in the range from 20 to 95% by weight, based on the total weight of the solution/mixture. In the case of starting materials which are sufficiently fluid under the reaction conditions, the hydrogenation can also be carried out in the absence of a solvent.

In addition to carrying out the reaction (hydrogenation) under anhydrous conditions, it has been found to be useful in a number of cases to carry out the reaction (hydrogenation) in the presence of water. The proportion of water can be, based on the mixture to be hydrogenated, up to 10% by weight, e.g. from 0.1 to 10% by weight, preferably from 0.2 to 7% by weight and in particular from 0.5 to 5% by weight.

The actual hydrogenation is usually carried out by a method analogous to the known hydrogenation processes as are described in the prior art mentioned at the outset. For this purpose, the starting material, e.g. the compound II, preferably as a liquid phase, is brought into contact with the catalyst in the presence of hydrogen. The catalyst can either be suspended in the liquid phase (suspension process) or the liquid phase is passed over a moving bed of catalyst (moving-bed process) or a fixed bed of catalyst (fixed-bed process). The hydrogenation can be carried out either continuously or batchwise. The process of the invention is preferably carried out as a fixed-bed process in trickle-bed reactors. The hydrogen can be passed over the catalyst either in cocurrent with or in countercurrent to the starting material to be hydrogenated.

Suitable apparatuses for carrying out a hydrogenation in the suspension mode and also for hydrogenation over a moving bed of catalyst or a fixed bed of catalyst are known from the prior art, e.g. from Ullmanns Enzyklopädie der Technischen Chemie, 4^{th} edition, Volume 13, p. 135 ff. and also from P. N. Rylander, “Hydrogenation and Dehydrogenation” in Ullmann's Encyclopedia of Industrial Chemistry, 5th ed. on CD-ROM.

The hydrogenation of the invention can be carried out either at a hydrogen pressure of one atmosphere or at a superatmospheric pressure of hydrogen, e.g. an absolute hydrogen pressure of at least 1.1 bar, preferably at least 10 bar. In general, the absolute hydrogen pressure will not exceed 325 bar and preferably 300 bar. The absolute hydrogen pressure is particularly preferably in the range from 20 to 300 bar, e.g. in the range from 50 to 280 bar.

The reaction temperatures in the process of the invention are generally at least 30° C. and will frequently not exceed a value of 200° C. In particular, the hydrogenation process is carried out at temperatures in the range from 40 to 150° C., e.g. from 40 to 100° C., and particularly preferably in the range from 45 to 80° C.

Possible reaction gases are hydrogen and also hydrogen-containing gases which comprise no catalyst poisons such as carbon monoxide or sulfur-containing gases, e.g. mixtures of hydrogen with inert gases such as nitrogen or offgases from a reformer, which usually further comprise volatile hydrocarbons. Preference is given to using pure hydrogen (purity≧99.9% by volume, particularly preferably ≧99.95% by volume, in particular ≧99.99% by volume).

Owing to the high catalyst activity, comparatively small amounts of catalyst, based on the starting material used, are required. Thus, less than 5 mol %, e.g. from 0.2 mol % to 2 mol %, of ruthenium will preferably be used per 1 mol of starting material in a suspension process carried out batchwise. When the hydrogenation is carried out continuously, the starting material to be hydrogenated will usually be passed over the catalyst in an amount of from 0.05 to 3 kg/(l(catalyst)·h), in particular from 0.15 to 2 kg/(l(catalyst)·h).

Of course, when the activity of the catalysts used in this process drops, they can be regenerated by the customary methods known to those skilled in the art for noble metal catalysts such as ruthenium catalysts. Mention may here be made of, for example, treatment of the catalyst with oxygen as described in BE 882 279, treatment with dilute, halogen-free mineral acids as described in U.S. Pat. No. 4,072,628, or treatment with hydrogen peroxide, e.g. in the form of aqueous solutions having a concentration of from 0.1 to 35% by weight, or treatment with other oxidizing substances, preferably in the form of halogen-free solutions. The catalyst is usually rinsed with a solvent, e.g. water, after the reactivation and before renewed use.

The hydrogenation process of the invention preferably comprises the complete hydrogenation of the aromatic rings of the bisglycidyl ether of the formula II 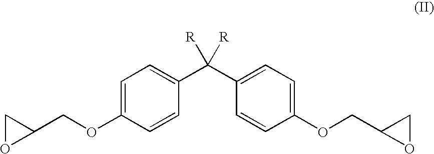
used, where R is CH₃ or H, with the degree of hydrogenation being >98%, particularly preferably >98.5%, very particularly preferably >99%, e.g. >99.3%, in particular >99.5%, e.g. in the range from >99.8 to 100%.

The degree of hydrogenation (Q) is defined by
Q(%)=([number of cycloaliphatic C6 rings in the product]/[number of aromatic C6 rings in the starting material])·100

The ratio, e.g. molar ratio, of the cycloaliphatic and aromatic C6 rings can preferably be determined by means of ¹H-NMR spectroscopy (integration of the aromatic and correspondingly cycloaliphatic ¹H signals).

The invention likewise provides bisglycidyl ethers of the formula I 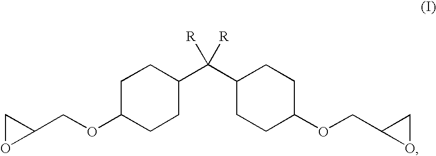
where R is CH₃ or H, which can be prepared by the hydrogenation process of the invention.

The bisglycidyl ethers of the formula I preferably have a content of corresponding oligomeric ring-hydrogenated ethers of the formula 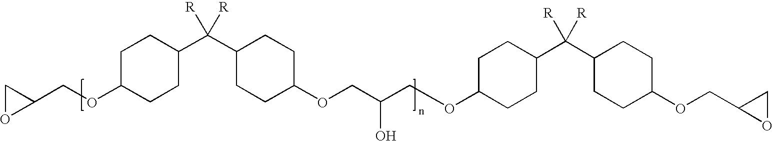
(where R is CH₃ or H) in which n=1, 2, 3 or 4, of less than 10% by weight, particularly preferably less than 5% by weight, in particular less than 1.5% by weight, very particularly preferably less than 0.5% by weight, e.g. in the range from 0 to <0.4% by weight.

The content of oligomeric ring-hydrogenated bisglycidyl ethers is preferably determined by heating the aromatic bisglycidyl ether for 2 hours at 200° C. and for a further 2 hours at 300° C., in each case at 3 mbar, or by GPC measurement (gel permeation chromatography).

As regards the further conditions for determining the oligomer content, see below.

The bisglycidyl ethers of the formula I preferably have a total chlorine content determined in accordance with DIN 51408 of less than 1000 ppm by weight, in particular less than 800 ppm by weight, very particularly preferably less than 600 ppm by weight, e.g. in the range from 0 to 400 ppm by weight.

The bisglycidyl ethers of the formula I preferably have a ruthenium content determined by mass spectrometry combined with inductively coupled plasma (ICP-MS) of less than 0.3 ppm by weight, in particular less than 0.2 ppm by weight, very particularly preferably less than 0.1 ppm by weight, e.g. in the range from 0 to 0.09 ppm by weight.

The bisglycidyl ethers of the formula I preferably have a platinum-cobalt color number (APHA color number) determined in accordance with DIN ISO 6271 of less than 30, particularly preferably less than 25, very particularly preferably less than 20, e.g. in the range from 0 to 18.

The bisglycidyl ethers of the formula I preferably have an epoxy equivalent weight determined in accordance with the standard ASTM-D-1652-88 in the range from 170 to 240 g/equivalent, particularly preferably in the range from 175 to 225 g/equivalent, very particularly preferably in the range from 180 to 220 g/equivalent.

The bisglycidyl ethers of the formula I preferably have a proportion of hydrolyzable chlorine determined in accordance with DIN 53188 of less than 500 ppm by weight, particularly preferably less than 400 ppm by weight, very particularly preferably less than 350 ppm by weight, e.g. in the range from 0 to 300 ppm by weight.

The bisglycidyl ethers of the formula I preferably have a kinematic viscosity determined in accordance with DIN 51562 of less than 800 mm²/s, particularly preferably less than 700 mm²/s, very particularly preferably less than 650 mm²/s, e.g. in the range from 400 to 630 mm²/s, in each case at 25° C.

The bisglycidyl ethers of the formula I preferably have a cis-cis:cis-trans:trans-trans isomer ratio in the range 44-63%:34-53%:3-22%.

The cis-cis:cis-trans:trans-trans isomer ratio is particularly preferably in the range 46-60%:36-50%:4-18%.

The cis-cis:cis-trans:trans-trans isomer ratio is very particularly preferably in the range 48-57%:38-47%:5-14%.

In particular, the cis-cis:cis-trans:trans-trans isomer ratio is in the range 51-56%:39-44%:5-10%.

The bisglycidyl ethers of the formula I are particularly preferably obtained by complete hydrogenation of the aromatic rings of a bisglycidyl ether of the formula II 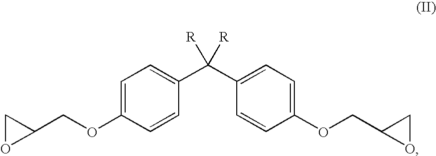
where R is CH₃ or H, with the degree of hydrogenation being >98%, particularly preferably >98.5%, very particularly preferably >99%, e.g. >99.3%, in particular >99.5%, e.g. in the range from >99.8 to 100%.

### EXAMPLES

1. Production of Catalysts 1 to 3 According to the Invention

A defined amount of the support material was placed in a dish and impregnated with 90-95% of the maximum amount of a solution of Ru(III) acetate (about 5% Ru in 100% acetic acid) in water which can be taken up by the support material. The following supports were selected:

silica gel extrudates (diameter (d)=1.5-4 mm, length (l)=to 10 mm) having an SiO₂ content of >99.5% by weight (0.3% by weight of Na₂O), a specific BET surface area of 100-200 m²/g, a water uptake (WU) of 0.85-1.0 g/g and a pore volume of 0.5-0.9 ml/g (DIN 66131), such as, e.g.

| Extrudate | | | Pore | | | |
|---|---|---|---|---|---|---|
| (d) | WU | BET | diameter** | | Pore volume** | |
| 1.5 mm | 0.95 g/g | 160-180 m²/g | 19 | nm | 0.8 | ml/g |
| 3 mm | 0.88 g/g | 110-125 m²/g | 23-24 | nm | 0.69-0.70 | ml/g |
| 4 mm | 0.89 g/g | 140-169 m²/g | 23 | nm | 0.84 | ml/g |

**Data from Hg sorption according to DIN 66134

C15 from Grace (BET surface area=181 m²/g, pore volume of 1.1 ml/g, Q₂/Q₃=13%, M(II):(Al(III)+Fe(II and/or III))=7.0), (M(II)=Ca(II)+Mg(II)), and

Davicat® S557 (Grade 57) from Grace-Davison (BET surface area=340 m²/g, pore volume of 1.1 ml/g, Q₂/Q₃=8.8%, M(II):(Al(III)+Fe(II and/or III))=4.6), (M(II)=Ca(II)+Mg(II)).

The material obtained in this way was in each case dried overnight at 120° C. The dried material was reduced for 2 hours at 300° C. in a stream of hydrogen at atmospheric pressure in a rotary sphere oven. After cooling and making the system inert (N₂), the catalyst was passivated with dilute air at room temperature. The reduced and passivated catalyst contained about 1.6-2% by weight of Ru, based on the total mass of the catalyst obtained.

TEM Analysis:

The ruthenium concentration within a catalyst particle decreases from the outside toward the interior, with an Ru layer having a thickness of about 200 nm being located at the particle surface. In the interior of the catalyst particle, the Ru particles have a size of up to about 2 nm. Beneath the ruthenium shell, aggregated and/or agglomerated Ru particles are observed in places. In this region, the size of the individual Ru particles is up to about 4 nm. Crystalline ruthenium was detected in the shell by means of SAD.

XRD analysis indicates a ruthenium crystallite size of about 8 nm.

The pore volume was determined by means of nitrogen sorption in accordance with DIN 66131.

The identification of the Qₙ structures (n=2, 3, 4) and the determination of the percentage ratio were carried out by means of solid-state ²⁹Si-NMR.
Qₙ=Si(OSi)ₙ(OH)₄₋ₙ where n=1, 2, 3 or 4.

Qₙ is found at −110.8 ppm when n=4, at −100.5 ppm when n=3 and at −90.7 ppm when n=2 (standard: tetramethylsilane) (Q₀ and Q₁ were not identified). The analysis was carried out under the conditions of “magic angle spinning” at room temperature (20° C.) (MAS 5500 Hz) with circular polarization (CP 5 ms) and using dipolar decoupling of ¹H. Owing to the partial superimposition of the signals, the intensities were determined by line shape analysis. The line shape analysis was carried out using a standard software package from Galactic Industries, with a “least square fit” being calculated iteratively.

Tabular Overview:

| | Catalyst 1 = cat. B from | | |
|---|---|---|---|
| | WO-A-02/100538 | Catalyst 2 based on | Catalyst 3 based |
| N₂ sorption: | (3 mm extrudates) | Davicat ® S557 | on C15 (Grace) |
| BET, m²/g | 117 | 341 | 181 |
| Pore diameter, nm | 24 | 11 | 19 |
| Pore volume, ml/g | 0.69 | 1.15 | 1.1 |
| Fe + Al, ppm*) | 400 | 125 | 47 |
| (Ca + Mg):(Fe + Al), | 0.1 | 4.6 | 7.0 |
| ppm/ppm*) | | | |
| ²⁹Si-NMR (MAS) | 30 | 9 | 13 |
| Q₂/Q₃, % | | | |

*)Oxidation states: Fe(II and/or III), Al(III), Ca(II), Mg(II).

The support of catalyst A from WO 02/100 538 corresponds to the support of catalyst B from WO 02/100 538 (same chemical composition), but the BET surface area is 68 m²/g and the pore volume is 0.8 ml/g.

To produce catalysts according to the invention, catalysts 1 to 3 are each impregnated with an Mg²⁺ salt solution, e.g. with an 80 mM (millimolar) aqueous Mg(NO₃)₂ solution, at room temperature for, for example, 15 minutes. The impregnated catalyst is rinsed with water and dried at 80° C.

2. Production of Catalysts A and B

Catalyst A (without Mg Impregnation, not According to the Invention)

The catalyst was produced by a method based on WO-A2-02/100 538.

Silica extrudates (diameter d=3 mm) having an SiO₂ content of >99.5% by weight (0.3% by weight of Na₂O), a pore volume of 0.7 ml/g (DIN 66131), a BET surface area of about 118 m²/g and a water uptake of 0.87 g/g of support were used. The support was placed in a dish and impregnated with an Ru acetate solution at 95% of the water uptake. The impregnated product was dried overnight at 120° C. The reduction was carried out for 2 hours at 300° C. in a stream of hydrogen at atmospheric pressure in a rotary sphere oven. After cooling and making the system inert (N₂), the catalyst was passivated with dilute air at room temperature. The catalyst obtained in this way was used as such (=catalyst A) or converted into catalyst B (see below).

Catalyst B (with Mg Impregnation, According to the Invention)

Catalyst A (20% by weight) was impregnated with 80% by weight of an 82.5 mM Mg solution (Mg(NO₃)₂.6H₂O) for 15 minutes at room temperature. The impregnated catalyst was rinsed with water and dried at 80° C.

3. Hydrogenation Examples

The conversion and degree of hydrogenation were determined by means of ¹H-NMR: sample weight: 20-40 mg, solvent: CDCl₃, 700 μliters using TMS as reference signal, sample tube: 5 mm diameter, 400 or 500 MHz, 20° C.; decrease in the signals of the aromatic protons versus increase in the signals of the aliphatic protons. The conversion reported in the examples is based on the hydrogenation of the aromatic groups.

The decrease in the epoxide groups was determined by comparison of the epoxide equivalent (EEW) before and after hydrogenation, in each case determined in accordance with the standard ASTM-D-1652-88.

The determination of ruthenium in the reaction mixture which had been freed of THF and water was carried out by mass spectrometry combined with inductively coupled plasma (ICP-MS, see below).

### Example 1

In a 300 ml autoclave, 150 g of a 30% strength by weight solution of 2,2-di[p-glycidoxyphenyl]propane (oligomer-containing standard product, ARALDIT GY 240 BD from Vantico, EEW=182) in THF were reacted with 3% by weight of water at 250 bar and 50° C. for 10 hours. 0.5 mol % (mol % of Ru based on 2,2-di[p-glycidoxyphenyl]propane) of the catalysts A and B were used in each case. (Batch method). After the reaction was complete, THF and water were separated off by distillation and conversion, selectivity and Ru content were determined.

| | | Selectivity [%] | |
|---|---|---|---|
| Catalyst | Conversion [%] | (EEW) | Ru content [ppm] |
| A | 89 | 94 (199) | 34 |
| B | 90 | 92 (203) | 2 |

The example shows that:
1. The Ru catalyst A is a high-performance hydrogenation catalyst for aromatic bisglycidyl ethers.
2. Impregnation of the catalyst with a magnesium salt (catalyst B) does not alter either the activity or selectivity but increases the stability considerably.

### Example 2 (comparison)

The reactor used was a heated reaction tube made of stainless steel (length: 0.8 m; diameter: 12 mm) which was charged with 75 ml of catalyst A and was provided with a circulation pump for the starting material and a separator with level control for sampling and regulation of the offgas.

A 30% strength by weight solution of 2,2-di[p-glycidoxyphenyl]propane (distilled product, EEW=171) in THF, which comprised 3% by weight of water, was initially fed to the hydrogenation. The hydrogenation was operated at a WHSV over the catalyst of 0.15 kg/l_{cat}·h, a feed/circulation ratio of 8, a temperature of 50° C. and a hydrogen pressure of 250 bar. The reactor was operated in the upflow mode.

After an operating time of 46 hours, a conversion of 95.4% at a selectivity of 69.3% (EEW=255) was achieved. The experimental run was stopped because of intensive Ru leaching (Ru content in the reaction product mixture, without THF and water: >4 ppm). (Ru leaching=removal of the noble metal from the support).

The example shows that:

When a supported Ru catalyst such as catalyst A is used in a continuous process, the catalyst displays leaching and is thus in need of improvement for an economic industrial process.

### Example 3

The reactor used was a heated reaction tube made of stainless steel (length: 0.8 m; diameter: 12 mm) which was charged with 75 ml of catalyst B and was provided with a circulation pump for the starting material and a separator with level control for sampling and regulation of the offgas.

A 30% strength by weight solution of 2,2-di[p-glycidoxyphenyl]propane (distilled product, EEW=172) in THF, which comprised 3% by weight of water, was initially fed to the hydrogenation. The hydrogenation was operated at a WHSV over the catalyst of 0.15 kg/l_{cat}·h, a feed/circulation ratio of 8, a temperature of 50° C. and a hydrogen pressure of 250 bar. The reactor was operated in the upflow mode.

After an operating time of 256 hours, 5 ppm by weight of Mg (based on bisglycidyl ether used (=BGE), calculated as 100%) in the form of Mg(NO₃)₂.H₂O were added to the feed.

After an operating time of 346 hours, the BGE concentration in the feed was increased to 40%, but the Mg concentration was maintained. The feed/circulation ratio was 11.

The conversions, selectivities and Ru concentrations achieved in the output from the reactor (without solvent) are shown in the following table.

| | Operating | Feed | Circulation | Conversion | Selectivity | Ruthenium | |
|---|---|---|---|---|---|---|---|
| Balance | time [h] | [g/h] | [g/h] | [%] | [%] | [ppm] | Remarks |
| 1 | 40 | 37.5 | 300 | 75.7 | 91.9 | <0.1 | |
| 2 | 64 | | | 81.7 | 90.7 | | |
| 3 | 88 | | | 82.2 | 89.8 | | |
| 4 | 112 | | | 85.6 | 89.9 | | |
| 5 | 136 | | | 85.7 | 89.9 | | |
| 6 | 160 | | | 86.4 | 90.4 | | |
| 7 | 184 | | | 86.1 | 90.4 | | |
| 8 | 208 | | | 86.8 | 89.5 | 0.4 | |
| 9 | 232 | | | 87.4 | 90.4 | 0.6 | |
| 10 | 256 | | | 83.3 | 90.3 | | Commencement of |
| | | | | | | | addition of Mg salt |
| 11 | 280 | | | 82.5 | 89.8 | ˜0.1 | |
| 12 | 304 | | | 79.4 | 90.2 | | |
| 13 | 328 | | | 79.3 | 90.2 | <0.1 | |
| 14 | 352 | | | 78.8 | 90.6 | | |
| 15 | 376 | 28.1 | | 78.3 | 88.8 | | Commencement of |
| | | | | | | | 40% strength feed |
| 16 | 400 | | | 81.5 | 88.9 | ˜0.1 | |
| 17 | 424 | | | 79.3 | 88.8 | | |
| 18 | 448 | | | 79.2 | 89.3 | | |
| 19 | 472 | | | 79.8 | 88.4 | | |
| 20 | 496 | | | 79.3 | 88.8 | <0.1 | |
| 21 | 520 | | | 79.6 | 89.3 | | Reduction of Mg |
| | | | | | | | addition to |
| | | | | | | | 2.5 ppm |
| 22 | 544 | | | 79.0 | 89.7 | | |
| 23 | 568 | | | 78.6 | 88.4 | 0.4 | |
| 24 | 592 | | | 77.8 | 89.2 | 0.3 | |
| 25 | 616 | | | 79.5 | 90.7 | | Reduction of Mg |
| | | | | | | | addition to |
| | | | | | | | 1.25 ppm |
| 26 | 640 | | | 78.9 | 90.2 | 0.9 | |
| 27 | 664 | | | 77.8 | 91.1 | | |
| 28 | 688 | | | 76.4 | 90.6 | 1.4 | |

The example shows that:
1. Impregnation with Mg leads, as also shown in the batch experiment, to the supported Ru catalyst becoming considerably more stable in continuous operation, too (balance 1-7).
2. After some time (˜208 hours), slight Ru leaching is observed (balance 8-9). The cause is presumably the washing out of the magnesium.
3. This can be countered by adding a small amount of Mg salt to the feed (10-14). The Ru content of the reaction product mixture can in this way be kept at <0.1 ppm.
4. Under these conditions a 40% strength by weight BGE solution can also be hydrogenated without problems (balance 15-20).
5. However, the amount of Mg additionally added is reduced to significantly below 5 ppm (2.5 or 1.25 ppm), slight (0.9 ppm) or strong (1.4 ppm) leaching is again observed (balance 21-28).
   4. Oligomer Content:

According to the invention, it has also been recognized that the oligomer content of the feed has an influence on the operating life of the catalyst: when a distilled feed (“low-oligomer” feed) is used, a slower catalyst deactivation than in the case of a standard commercial product (“oligomer-rich” feed) is observed. The oligomer content can be determined, for example, by GPC measurement (gel permeation chromatography):

| | | “Oligomers” | |
|---|---|---|---|
| | “Monomer” | 380-<520 | |
| Product | 180-<380 g/mol | g/mol | 520-1500 g/mol |
| Standard product | 89.98% by area | 2.05% by area | 7.97% by area |
| Distilled product | 98.80% by area | 0.93% by area | 0.27% by area |

Molar mass of 2,2-di[p-glycidoxyphenyl]propane: 340 g/mol

5. Description of the GPC Measurement Conditions

Stationary phase: 5 styrene-divinylbenzene gel columns “PSS SDV linear M” (each 300×8 mm) from PSS GmbH (Temperature: 35° C.).

Mobile phase: THF (flow: 1.2 ml/min.).

Calibration: MW 500-10 000 000 g/mol using PS calibration kit from Polymer Laboratories. In the oligomer range: ethylbenzene/1,3-diphenylbutane/1,3,5-triphenylhexane/1,3,5,7-tetraphenyloctane/1,3,5,7,9-pentaphenyldecane.

Evaluation limit: 180 g/mol.

Detection: RI (refractive index) Waters 410, UV (at 254 nm) Spectra Series UV 100.

The molar masses reported are, owing to different hydrodynamic volumes of the individual polymer types in solution, relative values based on polystyrene as calibration substance and are thus not absolute values.

The oligomer content determined in % by area determined by GPC measurement can be converted into % by weight by means of an internal or external standard.

GPC analysis of an aromatic bisglycidyl ether of the formula II (R═CH₃) used in the hydrogenation process of the invention displayed, for example, in addition to the monomer, the following contents of corresponding oligomeric bisglycidyl ethers:

Molar masses in the range 180-<380 g/mol: >98.5% by area,

in the range 380-<520 g/mol: <1.3% by area,

in the range 520-<860 g/mol: <0.80% by area and

in the range 860-<1500 g/mol: <0.15% by area.

6. Description of the Method for Determining the Evaporation Residue

About 0.5 g of each sample was weighed into a weighing bottle. The weighing bottles were subsequently placed at room temperature in a plate-heated vacuum drying oven and the drying oven was evacuated. At a pressure of 3 mbar, the temperature was increased to 200° C. and the sample was dried for 2 hours. The temperature was increased to 300° C. for a further 2 hours, and the samples were subsequently cooled to room temperature in a desiccator and weighed.

The residue (oligomer content) determined by this method on standard product (ARALDIT GY 240 BD from Vantico) was 6.1% by weight.

The residue (oligomer content) determined by this method on distilled standard product was 0% by weight. (Distillation conditions: 1 mbar, bath temperature 260° C., and temperature at which the distillate went over at the top 229° C.).

7. Determination of the Cis-Cis:Cis-Trans:Trans-Trans Isomer Ratios

A hydrogenated bisphenol A bisglycidyl ether (R═CH₃) product was analyzed by means of gas chromatography (GC and GC-MS). Here, 3 signals were identified as hydrogenated bisphenol A bisglycidyl ether.

A plurality of isomers can be formed by hydrogenation of the bisphenol A unit of the bisglycidyl ether. Depending on the arrangement of the substituents on the cyclohexane rings, cis-cis, trans-trans or cis-trans isomerism can occur.

To identify the three isomers, the products of the respective peaks were collected preparatively by means of a column arrangement. Each fraction was subsequently characterized by NMR spectroscopy (¹H, ¹³C, TOCSY, HSQC).

The preparative GC was carried out using a GC system having a column arrangement.

The sample was preseparated on a Sil-5 capillary (l=15 m, ID=0.53 mm, df=3 μm). The signals were cut to a 2nd GC column with the aid of a DEANS connection. This column served to check the quality of the preparative cut. Each peak was subsequently collected with the aid of a fraction collector. 28 injections of an about 10% strength by weight solution of the sample were prepared, corresponding to about 10 μg of each component.

Characterization of the isolated components was then carried out by NMR spectroscopy.

The determination of the isomer ratios of a hydrogenated bisphenol F bisglycidyl ether (R═H) was carried out analogously.

8. Determination of Ruthenium in the Ring-Hydrogenated Bisglycidyl Ether of the Formula I

The sample was diluted by a factor of 100 with a suitable organic solvent (e.g. NMP). The ruthenium content of this solution was determined by mass spectrometry combined with inductively coupled plasma (ICP-MS).

Instrument: ICP-MS spectrometer, e.g. Agilent 7500s

Measurement Conditions:

Calibration: External calibration in organic matrix

Atomizer: Meinhardt

Mass: Ru102

The calibration curve was chosen so that the required output value could be determined with certainty in the diluted measurement solution.

9. Determination of Chloride and Organically Bound Chlorine

The determination of chloride was carried out by ion chromatography.

Sample Preparation:

About 1 g of the sample was dissolved in toluene and extracted with 10 ml of high-purity water.

The aqueous phase was measured by means of ion chromatography.

Measurement Conditions:

Ion chromatography system: Metrohm

Precolumn: DIONEX AG 12

Separation column: DIONEX AS 12

Eluent: (2.7 mmol of Na₂CO₃+0.28 mmol of NaHCO₃)/liter water

Flow: 1 ml/min.

Detection: Conductivity after chemical suppression

Suppressor Metrohm module 753

50 mmol of H₂SO₄; high-purity water
(flow: about 0.4 ml/min.)
   Calibration: 0.01 mg/l to 0.1 mg/l

Coulometric determination of organically bound chlorine (total chlorine) in accordance with DIN 51408, part 2, “Bestimmung des Chlorgehalts”

The sample was burnt at a temperature of about 1020° C. in an oxygen atmosphere. The bound chlorine in the sample was in this way converted into hydrogen chloride. The nitrous gases, sulfur oxides and water formed in the combustion are removed and the combustion gas which has been purified in this way is fed into the coulometer cell. Here, the coulometric determination of the chloride formed is effected according to Cl⁻+Ag⁺→AgCl.
Sample weight range: 1 to 50 mg
Determination limit: about 1 mg/kg (substance-dependent)
Instrument: Euroglas (LHG), “ECS-1200”
Reference: F. Ehrenberger, “Quantitative organische Elementaranalyse”, ISBN 3-527-28056-1.

1. A heterogeneous ruthenium catalyst comprising silicon dioxide as support material, wherein the catalyst surface comprises alkaline earth metal ions (M²⁺) and the alkaline earth metal ions (M²⁺) are introduced into the catalyst surface by impregnating a preliminary heterogeneous ruthenium catalyst with a solution of an alkaline earth metal(II) salt.

2. The ruthenium catalyst according to claim 1, wherein the catalyst surface comprises magnesium ions (Mg²⁺).

3. The ruthenium catalyst according to claim 1, wherein the catalyst comprises from 0.1 to 10% by weight of ruthenium and the catalyst surface comprises from 0.01 to 1% by weight of the alkaline earth metal ion(s) (M²⁺), in each case based on the weight of the silicon dioxide support material.

4. The ruthenium catalyst according to claim 1, wherein the catalyst comprises from 0.2 to 5% by weight of ruthenium and the catalyst surface comprises from 0.05 to 0.5% by weight of the alkaline earth metal ion(s) (M²⁺), in each case based on the weight of the silicon dioxide support material.

5. The ruthenium catalyst according to claim 1, wherein the catalyst is produced by single or multiple impregnation of the support material with a solution of a ruthenium(III) salt, drying and reduction.

6. The ruthenium catalyst according to claim 1, wherein the solution of an alkaline earth metal(II) salt is an aqueous solution of magnesium nitrate or calcium nitrate.

7. The ruthenium catalyst according to claim 1, wherein the support material based on amorphous silicon dioxide has a BET surface area (in accordance with DIN 66131) from 30 to 700 m²/g.

8. The ruthenium catalyst according to claim 1, wherein the catalyst comprises less than 0.05% by weight of halide as determined by ion chromatography, based on the total weight of the catalyst.

9. The ruthenium catalyst according to claim 1, wherein the ruthenium is concentrated as a shell at the catalyst surface.

10. The ruthenium catalyst according to claim 9, wherein the ruthenium in the shell is partially or fully crystalline.

11. The ruthenium catalyst according to claim 1, wherein the alkaline earth metal ions are highly dispersed in the catalyst surface.

12. The heterogeneous ruthenium catalyst according to claim 1, wherein the percentage ratio of the signal intensities of the Q₂ and Q₃ structures Q₂/Q₃ in the silicon dioxide support material determined by means of solid-state ²⁹Si-NMR is less than 25.

13. The ruthenium catalyst according to claim 1, wherein the total concentration of Al(III) and Fe(II and/or III) in the silicon dioxide support material is less than 300 ppm by weight.

14. A process for hydrogenating a carbocyclic aromatic group to form the corresponding carbocyclic aliphatic group, comprising contacting the carbocyclic aromatic group with a heterogeneous ruthenium catalyst, wherein the catalyst comprises silicon dioxide as support material, and the catalyst surface comprises alkaline earth metal ions (M²⁺) and the alkaline earth metal ions (M²⁺) are introduced into the catalyst surface by impregnating a preliminary heterogeneous ruthenium catalyst with a solution of an alkaline earth metal(II) salt.

15. The process according to claim 14, wherein the carbocyclic aromatic group is a benzene ring to form the corresponding carbocyclic 6-membered ring.

16. The process as claimed in claim 15 for preparing a bisglycidyl ether of the formula I 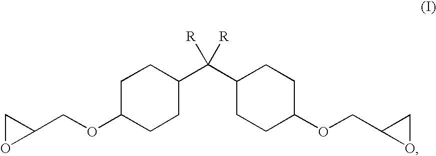
where R is CH₃ or H, by ring hydrogenation of the corresponding aromatic bisglycidyl ether of the formula II

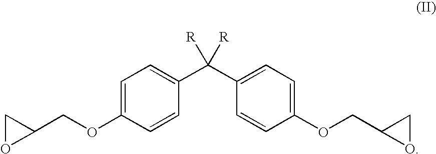

17. The process according to claim 16, wherein the aromatic bisglycidyl ether of the formula II has a content of corresponding oligomeric bisglycidyl ethers of less than 10% by weight.

18. The process according to claim 16, wherein the aromatic bisglycidyl ether of the formula II has a content of corresponding oligomeric bisglycidyl ethers of less than 5% by weight.

19. The process according to claim 18, wherein the oligomeric bisglycidyl ethers have a molecular weight in the range from 568 to 1338 g/mol for R═H and a molecular weight from 624 to 1478 g/mol for R═CH₃.

20. The process according to claim 14, wherein the hydrogenation is conducted at a temperature from 30 to 200° C.

21. The process according to claim 14, wherein the hydrogenation is conducted at absolute hydrogen pressures from 10 to 325 bar.

22. The process according to claim 14, wherein the hydrogenation is conducted over a fixed bed of catalyst.

23. The process according to claim 14, wherein the hydrogenation is conducted in a liquid phase in which the catalyst is comprised of a suspension.

24. The process according to claim 16, wherein the aromatic bisglycidyl ether of the formula II is used as a solution in an organic solvent which is inert toward the hydrogenation with the solution comprising from 0.1 to 10% by weight, based on the solvent, of water.

25. The process according to claim 14, wherein the solution of the aromatic bisglycidyl ether of the formula II to be hydrogenated comprises alkali earth metal ions (M²⁺).

26. The process according to claim 14, wherein the solution of the aromatic bisglycidyl ether of the formula II to be hydrogenated comprises magnesium ions (Mg²⁺).

27. The process according to claim 25, wherein the alkaline earth metal ion content of the solution is from 1 to 100 ppm by weight.

28. The process according to claim 25, wherein the alkaline earth metal ion content of the solution is from 2 to 10 ppm by weight.

29. The process according to claim 27 for preparing a bisglycidyl ether of the formula I. 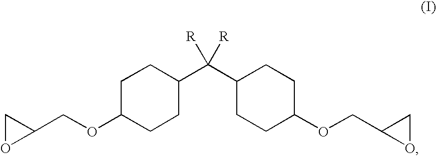
where R is CH₃ or H, which have a content of corresponding oligomeric ring-hydrogenated bisglycidyl ethers of the formula 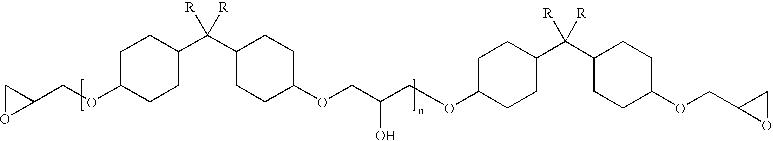
where n=1, 2, 3 or 4, of less than 10% by weight.

30. The process according to claim 29, wherein the bisglycidyl ether of the formula I has a content of corresponding oligomeric ring-hydrogenated bisglycidyl ethers of less than 5% by weight.

31. The process according to claim 29, wherein the bisglycidyl ether of the formula I has a content of corresponding oligomeric ring-hydrogenated bisglycidyl ethers of less than 1.5% by weight.

32. The process according to claim 29, wherein the bisglycidyl ether of the formula I has a content of corresponding oligomeric ring-hydrogenated bisglycidyl ethers of less than 0.5% by weight.

33. The process according to claim 29, wherein the content of oligomeric ring-hydrogenated bisglycidyl ethers is determined by heating the aromatic bisglycidyl ether for 2 hours at 200° C. and for a further 2 hours at 300° C., in each case at 3 mbar.

34. The process according to claim 29, wherein the content of oligomeric ring-hydrogenated bisglycidyl ethers is determined by gel permeation chromatography (GPC).

35. The process according to claim 34, wherein the content of oligomeric bisglycidyl ethers in % by area determined by GPC measurement is equated to a content in % by weight.

36. The process according to claim 29, wherein the bisglycidyl ether of the formula I has a total chlorine content determined in accordance with DIN 51408 of less than 1000 ppm by weight.

37. The process according to claim 29, wherein the bisglycidyl ether of the formula I has a ruthenium content determined by mass spectrometry in combination with inductively coupled plasma (ICP-MS) of less than 0.3 ppm by weight.

38. The process according to claim 29, wherein the bisglycidyl ether of the formula I has a platinum-cobalt color number (APHA color number) determined in accordance with DIN ISO 6271 of less than 30.

39. The process according to claim 29, wherein the bisglycidyl ether of the formula I has an epoxy equivalent weight determined in accordance with the standard ASTM-D-1652-88 from 170 to 240 g/equivalent.

40. The process according to claim 29, wherein the bisglycidyl ether of the formula I has a proportion of hydrolyzable chlorine determined in accordance with DIN 53188 of less than 500 ppm by weight.

41. The process according to claim 29, wherein the bisglycidyl ether of the formula I has a kinematic viscosity determined in accordance with DIN 51562 of less than 800 mm²/S at 25° C.

42. The process according to claim 29, wherein the bisglycidyl ether of the formula I has a cis-cis:cis-trans:trans-trans isomer ratio in the range 44-63%:34-53%:3-22%.

43. The process according to claim 42, wherein the bisglycidyl ether is obtained by complete hydrogenation of the aromatic rings of a bisglycidyl ether of the formula II 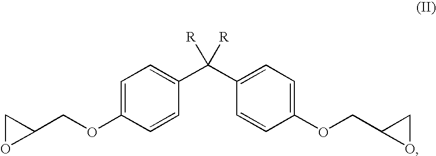
where R is CH₃ or H, with the degree of hydrogenation being >98%.
